(19) United States
(12) Patent Application Publication
(10) Pub. No.: US 2008/0299599 A1
(43) Pub. Date: Dec. 4, 2008

(54) FLUORESCENT PROTEINS FOR MONITORING INTRACELLULAR SUPEROXIDE PRODUCTION

(76) Inventors: Robert Dirksen, Pittsford, NY (US); Heping Cheng, Baltimore, MD (US); Shey-Shing Sheu, Pittsford, NY (US); Wang Wang, Ann Arbor, MI (US); Linda Groom, Lyons, NY (US)

Correspondence Address: BLANK ROME LLP, 600 NEW HAMPSHIRE AVENUE, N.W., WASHINGTON, DC, 20037, US

(21) Appl. No.: 11/851,148

(22) Filed: Sep. 6, 2007

Related U.S. Application Data
(60) Provisional application No. 60/842,660, filed on Sep. 7, 2006.

(51) Int. Cl. C12Q 1/02; C07K 14/00; C12N 15/11; C12N 5/06; A01K 67/027

(52) U.S. Cl. 435/29; 530/350; 536/23.1; 435/325; 800/13

(57) ABSTRACT

Protein probes and methods for measuring real-time changes in intracellular superoxide formation are provided. The probes include superoxide sensitive variants of yellow fluorescent and green fluorescent proteins. The probes, or nucleic acids encoding the probes, may be delivered to cells or organisms. Changes in the fluorescence of the probes may then be detected using standard real-time fluoroscopy techniques.

### STATEMENT OF PRIORITY

This application claims priority to U.S. Provisional Patent Application Ser. No. 60/842,660, filed Sep. 7, 2006 whose disclosure is hereby incorporated by reference herein.

### GOVERNMENT INTEREST

The subject matter of this application was made with support from the United States Government under Grant No. AR44657 from the National Institutes of Health. The United States Government may retain certain rights.

### FIELD OF THE INVENTION

The present invention relates to methods of monitoring the real-time production of superoxide in a cell or a compartment of a cell. The present invention also relates to modified proteins that are used to monitor the real-time superoxide production of a cell or a compartment of a cell.

### BACKGROUND OF THE INVENTION

Reactive oxygen species (ROS) are produced by cells in response to stress and in the course of aerobic metabolism. ROS are capable of causing damage to almost all of the molecular components of the cell, including lipids, fatty acids, amino acids, proteins and nucleic acids. Because of their ability to cause widespread damage, ROS are implicated in the development of a variety of disorders including ischemia-reperfusion injury, neurodegeneration, tissue inflammation, hypertension, atherosclerosis, diabetes and cancer. As changes in the cellular redox state caused by ROS accompany such an eclectic assortment of different types of human disease, interventions designed to combat oxidative stress (e.g. antioxidants) represent an intriguing class of therapeutic agents.

Superoxide (O2.−) is a widely produced, highly toxic radical anion that gives rise to other forms of ROS. Superoxide is produced as a side product of the reduction of molecular oxygen during energy production by the mitochondrial electron transport chain (ETC) and of the conversion of hypoxanthine to xanthine by the xanthine oxidase. Superoxide is also produced by NADPH oxidase in phagocytic leukocytes to destroy foreign pathogens. Because it is one of the main ROS produced in cells and because it gives rises to other species of ROS, there is a need in the art for methods of detecting superoxide and preventing its accumulation.

As mitochondria serve as the primary source for cellular energy production and generation of ROS, they play a critical role in disease development. Excessive increases in mitochondrial ROS trigger the opening of the mitochondrial permeability transition pore (mPTP) leading to apoptotic or necrotic cell death (Wang, Genes Dev. 15:2922-33, 2001; Brookes et al., Am. J. Physiol Cell Physiol. 287:C817-33, 2004). Paradoxically, physiological levels of mitochondrial ROS production also exert beneficial effects, and are required for some forms of cell signaling (Droge, Physiol Rev. 82:47-95, 2002).

Because of the wide impact that superoxide and other ROS have on cellular processes, several methods have been developed for measuring the oxidative/reductive, or redox, capacity of cells. Most of the current methods for measuring redox capacity involve the use of small molecule indicators, such as 5-(6)-chloromethyl-2′-7′-dichlorohydrofluorescene diacetate (DCFDA) (Reynolds and Hastings, J. Neurosci., 15:3318-27, 1995; Aon et al., J. Biol. Chem. 278:44735-44, 2003; Xi et al., Circ. Res. 97:354-62, 2005). Measurements made with DCFDA are non-ratiometric—meaning that ratios of emissions from different wavelengths cannot be compared—and exhibit substantial photobleaching and photocytoxicity. Further, measuring the redox environment of cells with small molecule indicators is a labor intensive process that typically requires that cells be harvested prior to obtaining readings. The time delays and disruptions to the cell's environment that occur during cell harvesting make it difficult to obtain an accurate reading of the in vivo redox environment, and make it impossible to monitor changes in the redox environment of a single cell over prolonged periods of time.

One solution to the problems associated with small molecule redox indicators has been to develop redox sensitive proteins. A green fluorescent protein (GFP) variant that is sensitive to the redox environment of cells is described in U.S. Patent Application Publication No. 2004/017112 to Remington, et al., which is hereby incorporated by reference herein. Although the redox sensitive GFP proteins described by Remington are an advancement over the small molecule based techniques described above, they have substantial disadvantages. One disadvantage is that the most significant signal changes indicated by the proteins described by Remington are through a loss of signal during oxidation, making it difficult to distinguish changes in redox environment because the signal to noise ratio is decreased. Further, the signal of the redox sensitive proteins described by Remington develops over the course of minutes or longer, precluding the possibility of real-time monitoring and witnessing transient redox events.

There remains the need in the art for redox sensing reagents that allow for facile real-time monitoring of the intracellular production of superoxide and other ROS.

### SUMMARY OF THE INVENTION

It is an object of the present invention to provide a method for the real-time monitoring of the formation of superoxide in a cell or specific cell compartment. The method of the invention uses a ratiometric protein probe for detection of formation of superoxide on a millisecond timescale making true real-time monitoring possible. The invention may be practiced with standard fluorescence microscopy techniques and equipment. The invention also allows the continuous monitoring of superoxide formation in cells while in culture.

It is a further object of the present invention to provide proteins capable of acting as real-time superoxide detecting probes. These proteins may be modified by standard genetic techniques to include targeting sequences that allow for their localization to a specific cell compartment. Upon localization of the superoxide sensing protein to the cell compartment, superoxide formation the cell compartment may be monitored in real-time.

It is a still further object of the present invention to provide a method for testing antioxidant agents. As the in vivo formation of superoxide can be monitored by the method of the invention, potential antioxidant agents can be added to cells and their effect on the formation of superoxide inside the cells can be monitored.

It is a further object of the present invention to provide a biomarker for diagnosis of a disease state. Proteins capable of monitoring superoxide formation within a cell can be expressed in disease models, and variations in superoxide formation can be monitored during the progression of the disease. As such, specific patterns of superoxide formation within a cell can be developed and correlated to the onset of specific diseases, allowing for the early diagnosis of a disease.

It is yet a further object to provide a research animal, such as a transgenic mouse, expressing a protein capable of monitoring changes in superoxide formation within a cell. These research animals could be crossed with like animals modeling a specific disease state, such as cancer or neurological disease. The resultant offspring would then be a disease model that allowed for monitoring of superoxide formation within the animal. Such an animal model would allow for in depth study of cellular changes in superoxide formation as a biomarker in the cellular environment during the progression of the disease.

### BRIEF DESCRIPTION OF THE FIGURES

FIG. 1: Circularly permuted yellow fluorescent protein (cpYFP) as a superoxide indicator. a, Excitation and emission spectra for fully reduced (10 mM reduced DTT) and fully oxidized cpYFP (1 mM aldrithiol) purified using the E. Coli. expression system. Ex: Excitation spectra obtained at 515 nm emission; Em: Emission spectra at 488 nm excitation. A redox-independent isosbestic point was identified near 405 nm excitation, permitting ratiometric measurement via dual wavelength excitation (488 nm/405 nm). b, The increase of cpYFP fluorescence emission (at 488 nm excitation) and its partial reversal by Cu/Zn—SOD (600 U/ml) when reduced cpYFP was exposed to xanthine (2 mM) plus xanthine oxidase (20 mU) under aerobic conditions. c, cpYFP signal was insensitive to H₂O₂ (0.1 and 10 mM) and slightly decreased by .OH (produced by the Fenton reaction with 1 mM H₂O₂ plus 0.1 mM FeSO4 under anaerobic conditions).

FIG. 2: cpYFP responses to peroxynitrite (a), nitric oxide (b), Ca2+ (c) pH (d) and several metabolites (e-g), including ADP (1 mM), ATP (10 mM), NAD+ (10 mM), NADH (1 mM), NADP+ (10 mM), and NADPH (1 mM). Peroxynitrite was produced by dissolve SIN-1 (1 mM) in aerobic solution and nitric oxide was produced by dissolve SIN-1 (1 mM) in anaerobic solution.

FIG. 3: Superoxide flashes in single mitochondria. a, Confocal visualization of a single mitochondrion superoxide flash in a rat cardiac myocyte. Upper panel: Confocal image of a mt-cpYFP expressing cardiac myocyte. The enlarged view shows dual-wavelength excitation (488 and 405 nm) imaging of the superoxide flash at 2 s intervals. The area shown is of 2.2×1.7 μm². b, Time course of the superoxide flash shown in a. c-d, Depression of superoxide flash frequency (c) and amplitude (ΔF/F₀, d) by the SOD mimetics, MnTMPyP (50 μM) and tiron (1 mM). n=12-64 flashes from 15-16 cells; *, P<0.05; #, P<0.01; †, P<0.001 versus control. e, Superoxide flashes in primary cultured hippocampal neurons. Arrows mark a spaghetti-shaped mitochondrion undergoing repetitive superoxide flashes. Images correspond to the designated time points. f, Frequencies of spontaneous superoxide flash activity in different cell types. n=21-53 cells.

FIG. 4: Characteristics of superoxide flashes in four different cell types. ΔF/F₀, amplitude; Tₚ, time to peak; T₅₀, 50% decay time after the peak. n=21-53 cells.

FIG. 5: Effect of scanning laser intensity on superoxide flash incidence. Average flash frequency at normal (1-2% transmission) or 5-fold higher laser intensity (5-10% transmission). n=12-18 cells.

FIG. 6: Expression of the pH biosensor, mt-EYFP, failed to detect flash-like events. (a) Mitochondrial expression pattern of mt-EYFP in cardiac myocytes; (b) Responses of mt-EYFP to extracellular application of NH₄Cl (30 mM) and subsequently FCCP (40 μM). (c) Frequency of mtcpYFP flashes and the lack of flash-like mt-EYFP events. n=12 cells.

FIG. 7: Cysteine-null mt-cpYFP variants (C171A/C193A and C171M/C193M) were insensitive to the oxidant aldrithiol (1 mM). Top panel shows the response of mt-cpYFP per se. Note also the low basal fluorescence in cardiac myocytes expressing the cysteine-null variants. Similar results were observed in at least 15 cells.

FIG. 8: Opening of mPTP underlies superoxide flash production. a, Colocalization of the ΔΨₘ indicator TMRM and the superoxide indicator mt-cpYFP in cardiac mitochondria revealed by triwavelength excitation imaging. b-c, Two types of ΔΨₘ depolarization were distinguished by the presence and absence of concurrent superoxide flashes, suggesting that distinct mechanisms may underlie flash-linked and flash-free ΔΨₘ oscillations. n=83 events from 19 cells. d, Loss of mitochondrial rhod-2 fluorescence at the onset of a superoxide flash (n=8 cells). e, Opposing effects of mPTP activation by atractyloside (20 μM, n=5 cells) and inhibition by bongkrekic acid (BA, 100 μM, n=16 cells) or cyclosporin A (1 μM, n=15 cells) on the properties of superoxide flashes. Tₚ, time to peak; T₅₀, 50% decay time. *, P<0.05; †, P<0.001 versus control.

FIG. 9: Mitochondrial ETC activity is an intrinsic regulator of superoxide flash incidence. a-f, Absence of superoxide flashes in 143B cells that are completely devoid of mitochondrial DNA (ρ° 143B). Superoxide flashes accompanying loss of TMRM signal were readily observed in wild type 143B TK-human osteosarcoma cells (WT 143B) as shown by fluorescent traces (a) and representative temporal diaries of superoxide flash incidence (c), but not in ρ° 143B cells (b,d) in spite of the presence of ΔΨₘ fluctuations (b). Atractyloside (20 μM) cannot induce superoxide flashes in ρ° 143B cells (e). f, Statistics of superoxide flash frequency in WT and ρ° 143B cells. n=5-10 cells. g, Attenuated superoxide flash activity in ETC-deficient cells. Insert: Treatment of PC12 cells with ethidium bromide (EB, 200 ng/ml) to inhibit mitochondrial DNA replication for up to 60 days resulted in a time-dependent decrease in expression of the mitochondrial DNA-encoded cytochrome C oxidase subunit I (COX-1) (hence, referred to as ρ− PC12 cells). R&A: rotenone (5 μM) and antimycin A (5 μg/ml). n=16-46 cells. *, P<0.05 versus wild type (WT PC12) cells. #, P<0.01 versus cells without the ETC inhibitors. h, Inhibition of superoxide flash activity by rotenone (Rot, 5 μM), antimycine A (AA, 5 μg/ml) or NaCN (5 mM) in rat adult cardiac myocytes. n=7-16 cells. †, P<0.001 versus control.

FIG. 10: Superoxide flashes in hypoxia and reoxygenation. a, Two dimensional map of superoxide flashes in a cardiac cell. Light boxes mark locations of superoxide flashes detected during a 100 s-scan during hypoxia, and dark boxes mark active sites 5 min following reoxygenation. b, Temporal diaries of superoxide flash incidence in three representative cells during hypoxia (left) and reoxygenation (right). Each vertical tick denotes a flash event; data in the top row correspond to the cell in a. c, Averaged data showing superoxide flash frequency during hypoxia, 5 min, and 1 hr after reoxygenation in the absence or presence of diazoxide pretreatment (30 μM for 20 min prior to hypoxia). n=10-16 cells. *, P<0.05 versus all other groups; #, P<0.01 versus diazoxide group. d, Schematic model for the genesis of mitochondrial superoxide flashes. In this model, the mPTP opens stochastically in response to physiological ROS levels set by constitutive ROS production by the ETC. Opening of the mPTP causes loss of ΔΨₘ, dissipation of chemical gradients across the inner membrane, and mitochondrial swelling, which could permit exaggerated respiration and favor the diversion of more electrons to ROS generation. This simple model accounts for superoxide flash properties (e.g., requiring both ETC and mPTP activities, all-or-none behavior, sensitivity to SOD mimetics) and predicts that superoxide flashes are a biomarker of oxidative stress. OMM: outer mitochondrial membrane; IMS: inter membrane space; IMM: inner mitochondrial membrane.

### DETAILED DESCRIPTION OF THE INVENTION

The present invention provides a method and protein probe for the facile real-time detection of superoxide formation within a cell or cellular compartment. The method allows for the detection of changes in cellular superoxide formation on a millisecond timescale using common fluorescence microscopy techniques.

The present invention measures superoxide formation within a cell or cellular compartment. It is to be understood that all methods described herein for measuring superoxide formation within a cell are also applicable for measuring superoxide formation within a cellular compartment, such as the mitochondria, the endoplasmic reticulum, or the nucleus. Superoxide formation within a specific compartment can be measured by targeting the protein probes of the invention to that specific compartment. Such targeting can be accomplished by the addition of localization sequences.

### Protein Probes of the Invention

The invention describes protein probes for detection and monitoring superoxide formation within a cell. A preferred embodiment the protein probe of the invention is the protein probe represented by SEQ ID NO. 1. SEQ ID NO. 1 is a modification of the circularly permuted yellow fluorescent protein (YFP) described as ratiometric pericam in U.S. patent application 20050208624 to Miyawaki et al. and Nagai et al. (Proc. Natl. Acad. Sci., 98:3197-3202, 2001), which are hereby incorporated by reference herein. The YFP described in US 20050208624 is a circularly permuted version of the yellow fluorescent protein described by Miyawaki et al. (Proc. Natl. Acad. Sci., 96: 2135-2140, 1999), which is hereby incorporated by reference herein. The calcium binding (calmodulin) and transduction (M13-calmodulin binding domain from myosin light chain) domains were removed from the protein described in US 20050208624 to form the novel superoxide sensing probe of the invention.

The embodiment of the invention represented by SEQ ID NO. 1 is a protein superoxide probe referred to as cpYFP and having the following properties:

1) a superoxide sensitive excitation maximum wavelength of 488 nm;

2) a superoxide insensitive excitation wavelength (isobestic point) of 405 nm; and

3) an emission maximum wavelength of 515 nm.

The embodiment of the invention set forth in SEQ ID NO. 1 is circularly permuted and otherwise modified from the wild type GFP (wtGFP) sequence described by Tsien (Annual Rev. Biochem., 67:509-44, 1998) which is presented here as SEQ ID NO. 2. For the purposes of describing the invention, specific residues will be referred to as they are numbered in SEQ ID NO. 1. To illustrate the function of various residues within SEQ ID NO. 1, these residues are compared with residues in wtGFP and mutants thereof, such as YFP mutants. When discussing the function of a residue within the sequence of non-circularly permuted fluorescent proteins, residues will be numbered as they are in Tsien (Annual Rev. Biochem., 67:509-44, 1998) and the residue numbering system will be referred to as wtGFP (SEQ ID NO. 2).

Many modifications, mutations, deletions and additions to SEQ ID NO. 1 can be made without detracting from the function of the protein probe. However, it is preferred that specific residues be unchanged in certain embodiments of the protein probes. Preferred residues include, but are not limited to: D13, A28, G40, F68, L158, C160, G177, Y178, G179, L180, K181 and C182. Other embodiments of the protein probe of the invention may have variations in the residues listed, non-limiting examples of which are described below. It should be understood that substituting residues in the protein probe cpYFP (SEQ ID NO. 1) may cause changes in the emission and excitation properties of the probe listed above.

Residue D13 of SEQ ID NO. 1 may contribute to the ratiometric properties of the protein probe. This aspartic acid substitution was introduced by Nagai (Proc. Natl. Acad. Sci. USA, 98:3197-202, 2001) in the development of the “ratiometric pericam” Ca²⁺ sensing protein that is the basis for SEQ ID NO. 1. It is also contemplated that residue 13 of SEQ ID NO. 1 may be other residues that allow the probe to retain its superoxide sensing properties, for example, histidine.

Residues A28 and G40 of SEQ ID NO. 1 may improve the folding properties of the protein. These residues correspond to residues 163 and 175 in wtGFP (SEQ ID NO. 2), which were found by Nagai et al. (Nature Biotechnology, 20:87-90, 2002) to improve the folding of the fluorescent protein at 37° C. It is also contemplated that residues 28 and 40 of SEQ ID NO. 1 may be other residues that allow the probe to retain its superoxide sensing properties. For example, residue 28 may be valine and residue 40 may be serine.

Residue F68 of SEQ ID NO. 1 may be important for determination of the fluorescence wavelength. Residue 68 of SEQ ID NO. 1 corresponds to residue 203 in the wtGFP (SEQ ID NO. 2). Various substitutions at residue 203 in wtGFP (SEQ ID NO. 2) cause a red shift in the fluoresce of the protein from the green region to the yellow region of the visible light spectrum, forming a YFP. YFPs described in the literature have either a histidine, tyrosine or phenylalanine residue at position 203 of the wild type sequence (see Tsien, Annual Rev. Biochem., 67:509-44, 1998). It is preferred that residue 68 of SEQ ID NO. 1 be phenylalanine, however, it may also be tyrosine or histidine or another residue that allows for the protein probe to retain its superoxide sensing properties. For example, F68 may be mutated to threonine to form a green fluorescing protein.

Residue L158 of SEQ ID NO. 1 may improve the maturation of the protein probe into a fluorescent protein. Residue L158 of SEQ ID NO. 1 corresponds to residue 46 of wtGFP (SEQ ID NO. 2), which was shown by Nagai et al. (Nature Biotechnology, 20:87-90, 2002) to improve the formation of the fluorophore. It is contemplated that residue 158 of SEQ ID NO. 1 may be other residues that allow the probe to retain its superoxide sensing properties, for example, phenylalanine.

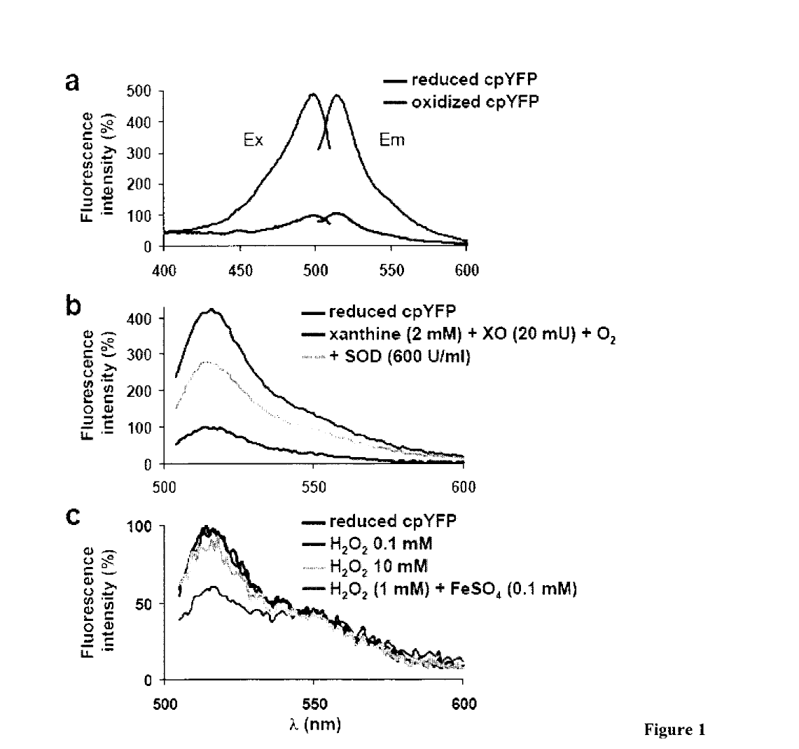

Residues C160 and C182 of SEQ ID NO. 1 may form the redox center of the protein probe. Substitution of both of these residues to either alanine (C160A/C182A) or methionine (C160M/C182M) completely abolishes the response of the probe to superoxide (See Example 2 and FIG. 1g), while substitution of only one of either C160 or C182 does not. As such, it is preferred that the probe of the invention of SEQ ID NO. 1 contain at least one of C160 or C182, or that the probe of the invention of SEQ ID NO. 1 contain both residues C160 and C182.

Residues G177, Y178 and G179 of SEQ ID NO. 1 may form the fluorophore of the cpYFP protein probe. Residues 177, 178 and 179 of SEQ ID NO. 1 correspond to residues 65, 66 and 67 in wtGFP (SEQ ID NO. 2). Residues 65, 66, and 67 of wtGFP (SEQ ID NO. 2) undergo a series of chemical reactions to form the fluorophore of the fluorescent protein family. In YFPs, residue 65 is typically glycine. As such, it is preferred that residue 177 of SEQ ID NO. 1 be glycine. However, other mutations within the fluorophore that retain the fluorescent properties of the protein probe are also contemplated. Non-limiting examples of such mutations include S177, T177, A177, W178, and H178.

Residues L180 and K181 of SEQ ID NO. 1 correspond to residues 68 and 69 of wtGFP (SEQ ID NO. 2) as originally introduced by Miyawaki et al. (Proc, Natl. Acad. Sci. USA, 96:2135-40, 1999). These residues were introduced in the non-circularly permuted protein to greatly reduce the pH sensitivity of YFP for detection of Ca²⁺. K69 of wtGFP (SEQ ID NO. 2) also has been shown to cause a further red shift in emission wavelength when introduced into a YFP (Miyawaki et al., Proc. Natl. Acad. Sci. USA, 96:2135-40, 1999). It is also contemplated that residues 180 and 181 of SEQ ID NO. 1 may be other residues that allow the probe to retain its superoxide sensing properties. For example, in certain embodiments, positions 180 and 181 may be mutated to valine and glutamine, respectively.

Along with being circularly permuted, the protein probe of SEQ ID NO. 1 also includes linker amino acid sequences not present in standard GFP or YFP sequences. In a preferred embodiment of the invention, these linker sequences are from residues 2 to 9 (RSGIGSAGY) and 104 to 112 (VDGGSGGTG), as shown in SEQ ID NO. 1. It is also contemplated that the linker sequences may be varied in any manner that retains the superoxide sensing properties of the protein probe. For example, the linker sequences may be shorter or longer. Further, it is contemplated that the size and relative hydrophobicity index of the amino acids in the linkers could be varied. Varying the types of the amino acids in the linker region may affect the flexibility of the protein and may cause other solvent effects or changes in the local pH surrounding the linker. For example, glycine linkers have been used to allow for greater flexibility in protein linkers (Mori et al., Science, 304:432-5, 2005). Even further, it is contemplated that one of the linker sequences may not be present at all. The amino acid sequence of the linker sequences can also vary greatly, as long as the superoxide sensing properties of the protein are maintained.

Preferably, the protein probe of the invention is a circularly permuted variant of YFP. However, in another embodiment of the invention, the protein probe may be the non-circularly permuted variant as provided in SEQ ID NO. 3, which may also be referred to as npYFP (non-permuted YFP).

Many modifications, mutations, deletions and additions to SEQ ID NO. 3 can be made without detracting from the function of the protein probe. However, it is preferred that specific residues be unchanged in embodiments of the protein probes. Preferred residues include, but are not limited to: D1177, A192, G204, F232, L75, C77, G94, Y95, G96, L97, K98 and C99. Other embodiments of the protein probe of the invention may have variations in the residues listed, non-limiting examples of which are described below. It should be understood that substituting residues in the protein probe npYFP may cause changes in the emission and excitation properties of the probe.

The preferred residues of npYFP (SEQ ID NO. 3) correspond to the preferred residues of cpYFP (SEQ ID NO. 1) described above. The corresponding residues are:

D177 of SEQ ID NO. 3 corresponds to D13 of SEQ ID NO. 1.

A192 of SEQ ID NO. 3 corresponds to A28 of SEQ ID NO. 1.

G204 of SEQ ID NO. 3 corresponds to G40 of SEQ ID NO. 1.

F232 of SEQ ID NO. 3 corresponds to F68 of SEQ ID NO. 1.

L75 of SEQ ID NO. 3 corresponds to L158 of SEQ ID NO. 1.

C77 of SEQ ID NO. 3 corresponds to C160 of SEQ ID NO. 1.

G94 of SEQ ID NO. 3 corresponds to G177 of SEQ ID NO. 1.

Y95 of SEQ ID NO. 3 corresponds to Y178 of SEQ ID NO. 1.

G96 of SEQ ID NO. 3 corresponds to G179 of SEQ ID NO. 1.

L97 of SEQ ID NO. 3 corresponds to L180 of SEQ ID NO. 1.

K98 of SEQ ID NO. 3 corresponds to K181 of SEQ ID NO. 1.

C99 of SEQ ID NO. 3 corresponds to C182 of SEQ ID NO. 1.

The residues listed above may have essentially the same function as their corresponding residues in SEQ ID NO. 1. Further, the non-limiting example mutations of the preferred residues of SEQ ID NO. 1 may also be substituted to for the preferred residues of SEQ ID NO. 3. In other words, as a non-limiting example, D177 of SEQ ID NO. 3 may also be histidine.

The protein probe of SEQ ID NO. 3 also includes similar linker amino acid sequences to those in SEQ ID NO. 1. In a preferred embodiment of the invention, these linker sequences are from residues 13 to 20 (RSGIGSAG) and 21 to 29 (VDGGSGGTG), as shown in SEQ ID NO. 3. It is also contemplated that the linker sequences may be varied in any manner that retains the superoxide sensing properties of the protein probe. For example, the linker sequences may be shorter or longer. Further, it is contemplated that the size and relative hydrophobicity index of the amino acids in the linker could be varied. Varying the types of the amino acids in the linker region may affect the flexibility of the protein and may cause other solvent effects or changes in the local pH surrounding the linker. For example, glycine linkers have been used to allow for greater flexibility in protein linkers (Mori et al., Science, 304:432-5, 2005). Even further, it is contemplated that one of the linker sequences may not be present at all. The amino acid sequence of the linker sequences can also vary greatly, as long as the superoxide sensing properties of the protein are maintained.

It should be noted that, although the non-circularly permuted version of a modified YFP is a functional superoxide sensing protein, this function is not inherent in other GFPs and YFPs. When a commercially available, mitchondrially targeted, non-circularly permuted YFP (Calbiochem, Mountain View, Calif.—catalog number 632347 (discontinued—now catalog number 632432)) was tested, it was found to have no superoxide sensing properties (data not shown).

Protein tags known in the art may be added to the protein probes to effect targeting, purification and/or location of the probes. One or more tags may be added to either the N- or C-terminus, or both termini, as required.

Various localization signals and targeting sequences that are well known in the art may be added to the probes as targeting tags. Targeting tags may be selected based on the intracellular compartment inside of which superoxide is to be monitored. For example, targeting tags may be added to probes to effect their targeting to the cytoplasm, the Golgi, the endoplasmic/sarcoplasmic reticulum, mitochondria, peroxisome and the nucleus, along with other cellular compartments. Non-limiting examples of sequences that may be used as targeting tags in the present invention are disclosed in Wickner and Schekman (Science, 310:1452-6, 2005) and Shaner et al. (Nature Methods, 2:905-09, 2005) which are hereby incorporated by reference herein.

Specific protein tags may be added to the probes of the invention to allow for their purification. Examples of protein tags that may be added to effect purification of the probes include, hexahistidine (His₆) tags, maltose binding protein (MBP) tags, glutathione-S-transferase (GST) tags, the IgG domain from protein A, and the like.

Specific protein tags may also be added to the probes of the invention to allow for their purification and/or localization after they are expressed inside a cell or cellular compartment. Examples of tags that may be added to effect location of the probes include hemagglutin (HA) tags, FLAG-tags, Myc-tags and the like. Protein probes bearing these tags can then be purified and/or identified using antibodies to the tags, as is well known in the art.

### Nucleic Acids of the Invention

The protein probes of the invention may be expressed from a nucleic acid sequence encoding the amino acid sequence of the probe. A preferred nucleic acid sequence of the invention is encoded by the nucleic acid sequence SEQ ID NO. 4, which is one of the possible nucleic acid sequences encoding the protein probe of SEQ ID NO. 1. Other nucleic acid sequences are contemplated by the invention, including other nucleic acid sequences encoding the probes of SEQ ID NO. 1 and SEQ ID NO. 3, along with nucleic acid sequences encoding other variants of protein probes, as described above.

The nucleic acid sequences of the invention may be incorporated into larger nucleic acids, such as a vector, to allow for their transformation into cells for expression of the protein probes. For example, the nucleic acid sequences of the invention may be incorporated into a vector that allows for transformation of the protein probes into mammalian cells, fungal cells or bacterial cells. The nucleic acid sequences may also be incorporated into viral vectors that allow for the transfection of mammalian or other types of cells.

If a protein tag is to be added to the probe, the nucleic acid sequence encoding the protein tag can be linked upstream or downstream from the nucleic acid sequence of the invention. As such, probes expressed from these nucleic acid sequences will contain the desired tags for targeting, localization, and the like. Further, it is also contemplated that the probe could be tagged to another cellular protein, such as xanthine oxidase or superoxide dimutase, predicted to influence superoxide production or degradation within the cell.

### Cell Lines and Organisms of the Invention

The invention contemplates cell lines stably or transiently expressing protein probes capable of monitoring intracellular superoxide formation. Nucleic acids encoding embodiments of the protein probe described above may be transfected or otherwise delivered to cells using methods known in the art. The nucleic acids encoding the protein probe will then be expressed during the regular growth of the cell line. Cell lines of the invention may be modified versions of mammalian, fungal, bacterial, insect, fish and plant cell lines. Non limiting examples of mammalian cells lines which may be modified include HeLa cells, MDCK cells, CHO cells, MCF-7 cells, U87 cells, A172 cells, HL60 cells, A549 cells, Vero cells, GH3 cells, 9L cells, MC3T3 cells, C3H-10T1/2 cells, C2C12 cells, PC12 cells, 143B cells and NIH-3T3 cells. Real-time changes in an intracellular superoxide formation in these cells can then be monitored by standard fluorescence techniques.

The invention also contemplates organisms that contain cells expressing protein probes capable of monitoring intracellular superoxide formation. Nucleic acids encoding embodiments of the protein probe described above may be incorporated into the DNA of the organism or delivered to cells as an extra-chromosomal element. After the nucleic acid encoding a protein probe is provided to at least some of the cells of an organism, these cells of the organism will express a superoxide sensitive protein probe. Any research model organism can be modified to express the protein probe of the invention, including, rats, mice, zebrafish, Caenorhabditis elegans, yeasts such as Saccharomyces cerevisiae, Schizosaccharomyces pombe and Pichia pastoris and bacteria such as Escherichia coli.

The modified organisms of the invention can then be used for monitoring intracellular superoxide formation under standard growth and development conditions. These organisms may also be exposed to a variety of agents, both therapeutic and toxic, to determine the effect of these agents on intracellular superoxide formation. Further, the modified organisms of the invention may be crossed with known disease organism models. As the progeny of these crosses will both develop the disease in question and express superoxide sensitive protein probes, they may be used to monitor the change in intracellular superoxide formation during the progression of the disease.

### Methods for Monitoring Intracellular Superoxide

The methods of monitoring superoxide formation in a cell or cellular compartment of the invention can be carried out using the standard techniques for expression and visualization of fluorescent proteins known in the art. Non-limiting examples of such techniques can be found in Silver (J. Biol. Chem., 277:34042-7, 2002) and Weiss et al. (Am. J. Physiol. Cell Physiol., 287:C1094-1102, 2004), which are hereby incorporated by reference herein.

### Monitoring the Effect of an Agent

Using the methods described above and other methods known in the art, the cell lines and organisms of the invention may be used to monitor the effect of an agent on intracellular superoxide formation. Agents that may be tested include therapeutic agents, such as pharmaceuticals and biologics, known toxic agents and agents with unknown effect. Such agents may be administered at levels previously known from pharmacological or toxicological studies.

After an agent is administered, the changes in superoxide formation may be monitored. Such changes will be indicative of the effects of the agent, and may be correlated with the development of a specific disease state by analyzing the pattern of change.

### Diagnosis of a Disease State

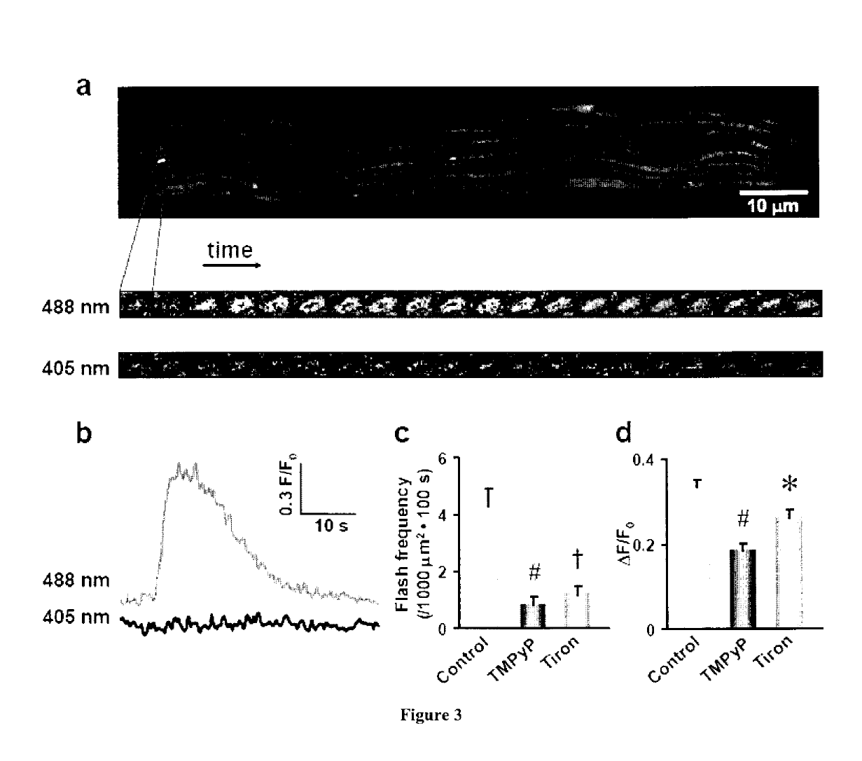

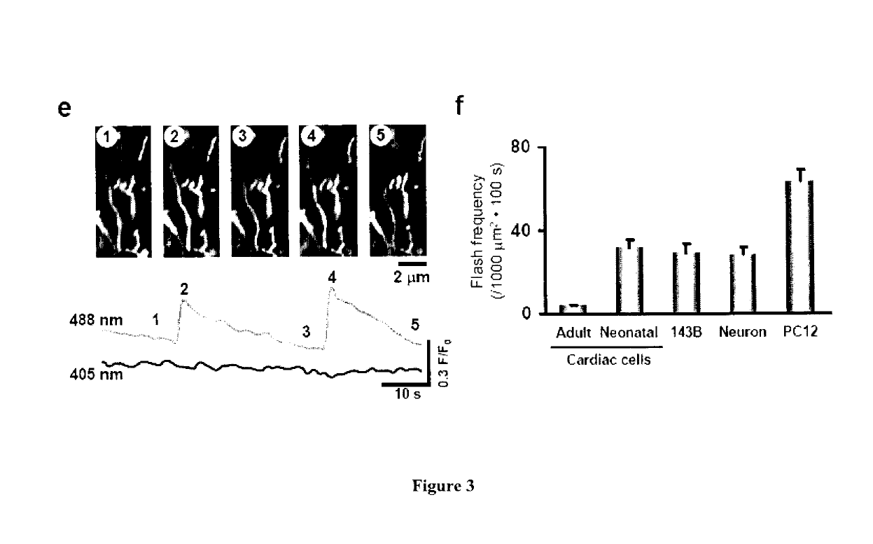

As changes in redox status are known to occur in many different disease states, the protein probes of the invention can be used as a biomarker for ischemia/reperfusion injury and protection from reperfusion injury by drug or ischemic preconditioning paradigms, as well as a marker for apoptosis, neurodegenerative disease, aging, diabetes, atherosclerosis, malignancies, infections and other ailments. Further examples of disease states that may be associated with the formation of superoxide and other ROS can be found in Droge (Physiol. Rev., 82:47-95, 2002). Each of these ailments could potentially be detected by changes in the cellular and/or subcellular superoxide formation, such as changes in the incidence and/or properties of transient changes in mitochondrial superoxide production (termed superoxide flashes, see FIG. 3).

Through repetitive studies using the test cell lines and organisms of the invention, specific patterns of change in superoxide formation may become apparent. These patterns may be used as biomarkers for predicting the onset of a particular disease state or the exposure to a specific agent. For example, in a model disease system, the incidence, properties and location of superoxide flashes could be observed in the model cells. Specific patterns of superoxide flashes may be observed that coincide with the onset (or progression) of the disease in the system. These patterns could then be used for predicting the onset of the disease in a patient.

### Methods for Transfecting Cells

Nucleic acids encoding the protein probes of the invention may be transfected into cells using methods known in the art. Non-limiting examples of transfection systems that may be used in conjunction with the present invention include the FuGENE® transfection system (Roche Applied Science, Indianapolis, Ind.) or the Lipofectamine™ 2000 system (Invitrogen, Carlsbad, Calif.). Other transfection methods are contemplated, including those that do not involve commercially prepared reagents, for example, nuclear cDNA injection as described by Weiss et al., (Am. J. Physiol. Cell Physiol., 287:C1094-1102, 2004).

The examples set forth below are meant to provide non-limiting examples of methods of the invention. It should be apparent that there are variations of the invention not presented in the examples below that fall within the scope and the spirit of the invention as claimed.

### EXAMPLES

### Example 1

### Materials and Methods

cDNA Constructs

mt-cpYFP was constructed from mitochondrial targeted ratiometric pericam (rpericamMT) cloned into pcDNA3 (Nagai et al., Proc. Natl. Acad. Sci. USA, 98: 3197-3202, 2001) by removing nucleotide sequences encoding calmodulin (nt 886-1323) and M13 (nt 49-126) using the gene splicing by overlap extension (SOE) technique (Horton et al, Gene, 77:61-68, 1989). The final PCR product was digested with HindIII/XbaI and cloned into the 5352 bp HindIII/XbaI fragment of pcDNA3. cpYFP was constructed from mt-cpYFP by removing nucleotide sequences encoding the 11 amino acid (LSLRQSIRFFK) mitochondrial targeting sequence of cytochrome oxidase subunit IV (nt 4-36) using gene-SOEing. The final PCR product was digested with HindIII/XbaI and cloned into the 5352 bp HindIII/XbaI fragment of pcDNA3. Double cysteine-to-alanine and cysteine-to-methionine substitutions in mt-cpYFP (C171A/C193A, and C171M/C193M) were constructed using a standard two-step site directed mutagenesis strategy. All sequences generated and modified by PCR were checked for integrity by sequence analysis. mt-EYFP was from Clontech.

Spectral Analysis of cpYFP

cpYFP cDNA (807 bp) was cloned into a prokaryotic expression vector (pRSET) and transferred into E. Coli cell line (BL21(DE3)LysS) for large-scale protein expression. In vitro redox calibration of cpYFP fluorescence was carried out using methods described previously (Hanson et al., J. Biol. Chem., 279: 13044-13053, 2004). Briefly, under an inert environment, purified cpYFP protein (1 μM) was incubated with either 10 mM reduced DTT or 1 mM aldrithiol for at least 3 hours, allowing for solution equilibration. Reduced DTT was removed from the solution allowing measurement of cpYFP response to various ROS and metabolites. The calibration solution contained (in mM): HEPES 75, KCl 125, and EDTA 1, pH=8.0. Emission and excitation spectra of reduced and oxidized cpYFP in the presence of designated reagents were obtained with a spectrofluorimeter (Model: CM1T101, HORIBA Jobin Yvon, Inc.) filled with nitrogen gas.

### Confocal Imaging

Enzymatically isolated rat ventricular myocytes and hippocampal neurons in primary culture were infected with adenovirus carrying the mt-cpYFP gene or its mutants at an m.o.i. of 1:100 and cultured for 2 to 3 days (Zhou et al., Am. J. Physiol. Heart Circ. Physiol., 279; H429-H436, 2000). Similar conditions were used when expressing mt-cpYFP in other cell types. To obtain spatially and temporally resolved fluorescent images of mt-cpYFP, a Zeiss LSM 510 confocal microscope equipped with a 63×, 1.3NA oil immersion objective and a sampling rate of 0.7 s/frame was used. Dual wavelength excitation imaging of mt-cpYFP was achieved by alternating excitation at 405 and 488 nm and collecting emission at >505 nm. Tri-wavelength excitation imaging of mt-cpYFP and TMRM (20 nM) or rhod-2 was achieved by tandem excitation at 405, 488, and 543 nm, and the emission was collected at 515-550, 515-550 and >560 nm, respectively. To increase mitochondrion retention of rhod-2, the indicator loading protocol described by Hajnoczky C et al. was used with modification (Hajnoczky et al., Cell, 82: 415-424, 2000). Briefly, cells were loaded with 4 μM rhod-2 AM (after NaBH₄ quenching) at 4° C. for 1 hr, and then changed to normal culture medium for 4 hrs. The standard extracellular perfusion solution contained (in mM): NaCl 137, KCl 4.9, CaCl₂1, MgSO₄ 1.2, NaH₂PO₄ 1.2, glucose 15, and HEPES 20 (pH 7.4). Digital image processing was performed using IDL software (Research Systems) and customer-devised programs.

### Mitochondrial DNA-Deleted or Deficient (ρ° or ρ−) Cells

ρ° 143B TK human osteosarcoma cells and its wild type control were a generous gift from Dr. Nadja C. de Souza-Pinto (National Institute on Aging, NIH). Wild type and ρ° 143B cells were cultured under identical conditions, in DMEM medium supplemented with 10% FBS, 100 μg/ml pyruvate, 100 μg/ml bromodeoxyuridine and 50 μg/ml uridine17. Mitochondria of ρ° 143B cells completely lack mitochondrial respiration, due to the loss of critical ETC proteins including constituents of complex I (ND1-6, ND4L), complex III (cytochrome b) and complex IV (COX I-III) encoded by mitochondrial DNA. To partially deplete mitochondrial DNA and allow partial disruption of mitochondrial respiration, PC12 pheochromocytoma cells were cultured in DMEM medium with 10% FBS, 200 ng/ml ethidium bromide, 100 μg/ml pyruvate and 50 μg/ml uridine for up to 60 days. Depletion of mitochondrial DNA was evidenced by western blot analysis of cytochrome C oxidase subunit I.

### Hypoxia and Reoxygenation Treatment of Cardiac Myocytes

Cardiac myocytes expressing mt-cpYFP were cultured in a hypoxia chamber (Billups-Rothenberg) at 37° C. and ventilated with 95% N2 plus 5% CO₂ for 6 hours. At the end of hypoxia treatment, culture dishes were sealed with a plastic cover and immediately transferred onto the stage of a confocal microscope. After recording superoxide flashes under hypoxic condition, reoxygenation was achieved by removing the seal and superfusing cells with standard oxygenated extracellular solution.

### Statistics

Data were reported as mean ±SEM. Paired and unpaired Student's t test and ANOVA with repeated measurements were applied, when appropriate, to determine statistical significance of the differences. P<0.05 was considered statistically significant.

### Example 2

### Spectral Analysis of cpYFP

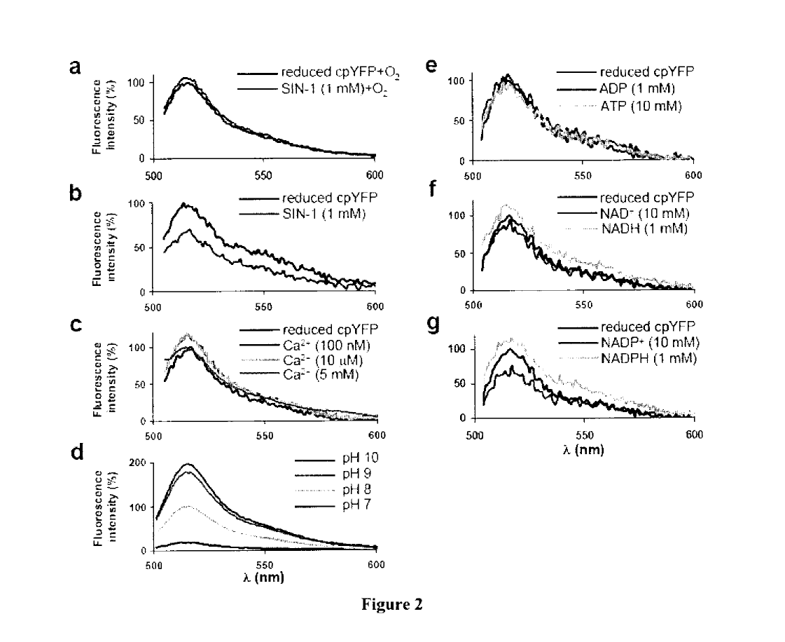

Unexpectedly, it was found that a circularly permuted yellow fluorescent protein (cpYFP), previously used to construct the Ca²⁺ indicator pericam (Nagai et al., Proc. Natl. Acad. Sci USA, 98: 3197-3202, 2001), can serve as a novel biosensor for superoxide anions (O₂.⁻), the primal ROS from the electron transfer chain (ETC) in mitochondria, via a redox dependent mechanism. Using cpYFP purified from an E. coli expression system, excitation and emission fluorescence spectra were measured in response to reducing (10 mM reduced DTT) and oxidizing manipulations (1 mM aldrithiol). The oxidized cpYFP was about five times brighter than the fully reduced species when excited at 488 nm (FIG. 1a), indicative of a good signal-to-background in contrast to recently reported redox-sensitive GFP probes (Hanson et al., J. Biol. Chem. 279: 13044-13053, 2004; Ostergaard et al., EMBO J, 20: 5836-5862, 2001). Extensive in vitro experiments were performed to determine the selectivity of cpYFP among physiologically relevant oxidants and metabolites. It was found that compared to the fully reduced state, cpYFP fluorescence displayed a 420% increase in response to O₂— produced by the xanthine/xanthine oxidase (2 mM/20 mU) system under aerobic conditions; addition of Cu/Zn-superoxide dismutase (600 U/ml) partially inhibited this response (FIG. 1b). The cpYFP signal, however, was insensitive to hydrogen peroxide (H₂O₂) over a wide range of concentrations (0.1-10 mM) (FIG. 1c) and peroxynitrite (FIG. 2), and was decreased by hydroxyl radicals (.OH) (FIG. 1c) and nitric oxide (FIG. 2). Other metabolites tested, including ATP, ADP, NAD(P)+, NAD(P)H and Ca²⁺ at physiological concentrations, exerted negligible or only marginal effects (FIG. 2). As would be expected of a fluorescent protein-based indicator (Nagai et al., Proc. Natl. Acad. Sci. USA, 98: 3197-3202, 2001; Belousov, et al., Nat. Methods, 3:281-286, 2006) cpYFP was brighter in basic environments such as those found within the mitochondrial matrix (pH 8.0) (FIG. 2).

### Example 3

### Expression of cpYFP in Cardiac Myocytes

Adenoviral gene transfer was employed to express cpYFP targeted to the mitochondria of cardiac myocytes via a cytochrome C oxidase subunit IV (COX IV) targeting sequence (mt-cpYFP).

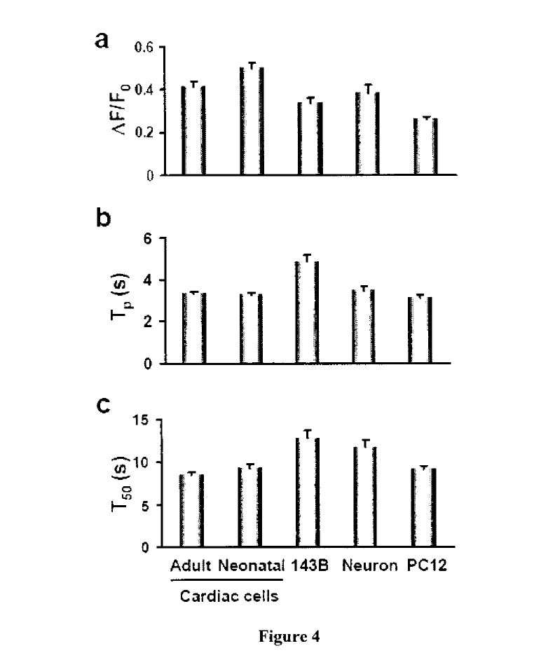

Confocal imaging revealed that mt-cpYFP stained bundle-like subcellular structures that were punctuated at Z-lines of the sarcomere, in agreement with spatial organization of cardiac mitochondria (FIG. 3a; Ramesh et al., Ann. N.Y. Acad. Sci., 853:341-344, 1998). Strikingly, it was found that localized flashes of mt-cpYFP fluorescence occur stochastically in a quiescent background (FIGS. 3a-b). A typical flash rose abruptly, peaked in 3.5±0.1 s, and then dissipated with a half time of 8.6±0.2 s (n=409) (FIGS. 3b and 4). The averaged fold-increase of mt-cpYFP fluorescence in a flash was 0.41±0.02 (ΔF/F0); the top 10% brightest events, which were most likely located on or close to the confocal imaging plane, displayed a ΔF/F0 of 1.0±0.1 (n=41). While randomly distributed throughout the myocyte, individual flashes were sharply confined to tiny elliptical areas each spanning 0.94±0.01 μm laterally and 1.68±0.03 μm longitudinally (n=409 flashes from 53 cells), while mitochondria in their immediate vicinity remained quiescent.

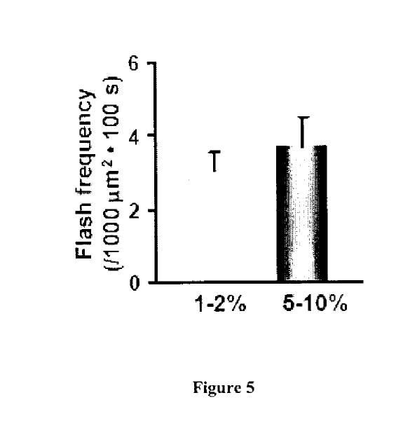

Since a 5-fold increase of the scanning laser intensity did not significantly alter the rate of flash production (FIG. 5), flashes described above were unlikely a phenomenon induced by photostimulation.

### Example 4

### Spontaneous mt-cpYFP Fluorescent Flashes Reflect Bursts of Matrix O₂.⁻ in Single Mitochondria (Superoxide Flashes) Under Physiological Conditions

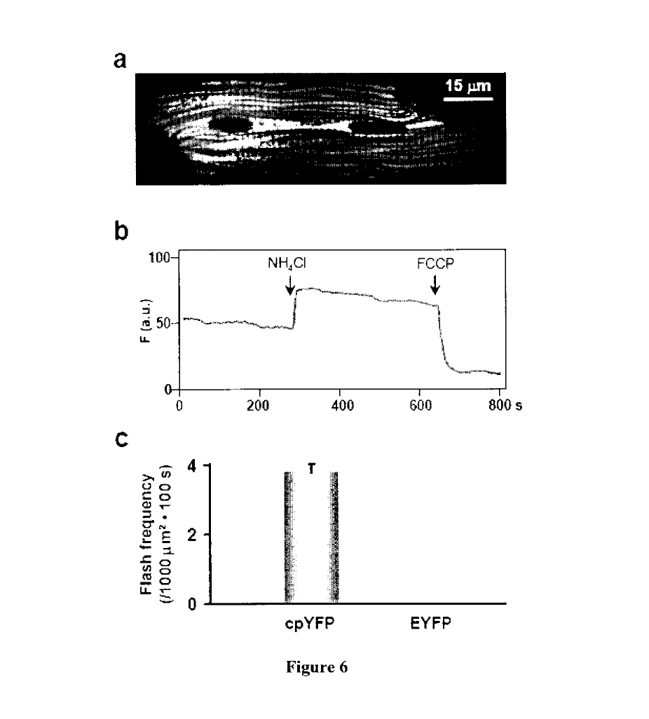

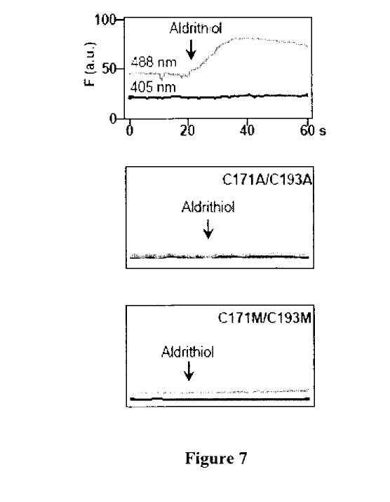

Experiments using mitochondrially targeted-EYFP as a pH biosensor (Takahashi et al., Biotechniques, 30: 804-808, 2001) failed to detect transient mitochondrial alkalinisation with a similar frequency and time course as flashes (FIG. 6), excluding mitochondrial alkalosis as an explanation for flashes. Importantly, application of MnTMPyP (50 μM), an SOD mimetic, inhibited flash activity by 83% and halved flash amplitude (ΔF/F₀=0.18±0.02, n=12; p<0.01 vs control) (FIGS. 3c-d); tiron (1 mM), a superoxide radical scavenger, similarly diminished the frequency and amplitude of flashes (FIGS. 3c-d), supporting their O₂.⁻ origin. Substituting the only two cysteine residues in cpYFP with either alanine (C171A/C193A) or methionine (C171M/C193M) diminished basal fluorescence and made the indicator redox-insensitive (FIG. 7). Mitochondrial flash activity was never detectable in cardiac cells expressing either of the two cysteine-null, redox-insensitive cpYFP variants (n=15 cells).

### Example 5

### Superoxide Flashes are not Unique to Cardiac Cells

Superoxide flashes were not unique to cardiac cells, but appeared to be universal among a wide diversity of cell types examined, including skeletal myotubes, neurons, neuroendocrine cells, fibroblasts and osteosarcoma cells. FIG. 3e shows superoxide flashes resolved in spaghetti-shaped mitochondria in primary cultured hippocampal neurons. Careful inspection revealed that multiple superoxide flashes can occur within one mitochondrion. The rate of superoxide flash occurrence, however, varied widely across cell type, ranging from 3.8±0.5 (n=53 cells) in adult cardiomyocytes, to 31±4 (n=24 cells) in primary cultured hippocampal neurons, and up to 63±6 (events per 1000 μm2 cell area per 100 s, n=37 cells) in PC12 pheochromocytoma cells (FIG. 3f). However, despite large variations in cellular and mitochondrial morphology, the fundamental properties (amplitude, time-to-peak, decay time) of individual superoxide flashes are highly comparable (FIG. 4), suggesting that a common or similar mechanism underlies mitochondrial superoxide flash generation. Real-time visualization of superoxide flashes thus uncovers a brief, intermittent mode of bursting superoxide anion production in mitochondria. Superoxide flashes thus represent elementary “digital” events of mitochondrial ROS metabolism and signaling.

### Example 6

### Mechanism for the Genesis of Superoxide Flashes

The temporal and spatial characteristics of mitochondrial superoxide flashes suggest that these events reflect a sudden, probabilistic transient excitation of the mitochondrial O₂.−-producing machinery. To this end, opening of the mitochondrial permeability transition pore (mPTP) by metabolic stress (Romashko et al., Proc. Natl. Acad. Sci. USA, 95: 1618-1623, 1998), photostimulation (Zorov et al., J. Exp. Med., 192: 1001-1014, 2000), excessive ROS or Ca²⁺ (Vercesi et al., Biosci. Rep., 17: 43-52, 1997; Duchen et al., Cell Calcium 28: 339-348, 2000) is known to stimulate ROS production while dissipating the mitochondrial membrane potential (ΔΨₘ) (Huser et al., Biophys. J., 74; 2129-2137, 1998) and permitting solute traffic (<1,000 Da) between the mitochondria matrix and the cytosol (Crompton, Biochem. J. 341: 233-249, 1999).

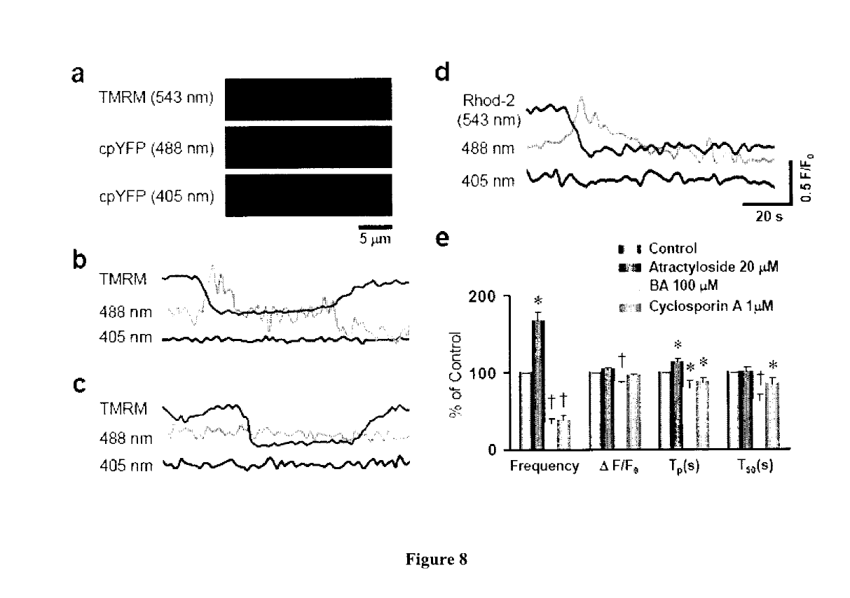

To test the hypothesis that superoxide flashes arise from stochastic activity of mPTP, cells were stained with TMRM, a ΔΨₘ indicator whose fluorescent signal is spectrally separable from that of mt-cpYFP (FIG. 8a). Simultaneous measurement of TMRM and mt-cpYFP signals revealed that every superoxide flash coincided with a decrease in ΔΨₘ (n=89 events from 19 cells; FIG. 5b), but not vice versa (FIG. 8c). The flash-linked ΔΨₘ flickers are consistent with mPTP activation in a flash; the fact that the vast majority (>80%) of ΔΨₘ flickers are flash-free supports the notion that not all ΔΨₘ flickers are related to mPTP opening (Zorov et al., J. Exp. Med., 192: 1001-1014, 2000, O'Reilly et al., Am. J. Physiol. Cell, Physiol. 286: C1139-1151). Using mitochondrion-entrapped rhod-2 (752 Da, commonly used as Ca²⁺ indicator), it was further demonstrated that the occurrence of superoxide flashes always coincided with a rapid and virtually irreversible loss of rhod-2 fluorescence (n=8, FIG. 8d), as if a significant portion of rhod-2 leaked out of the mitochondrion through mPTP opening.

To determine whether mitochondrial superoxide production can be tuned by altering mPTP activity, it was shown that inhibition of mPTP by bongkrekic acid (BA, 100 μM) markedly attenuated the incidence of superoxide flashes to 33% of control while reducing their amplitude and abbreviating their kinetics (FIG. 8e); similar results were also obtained with cyclosporine A (1 μM, FIG. 5e), a second mPTP inhibitor. Conversely, mPTP activation by atractyloside (20 μM) was sufficient to significantly augment superoxide flash frequency (FIG. 8e). These results corroborate that mPTP opening is a prerequisite for ignition of superoxide flashes, and indicate that superoxide flashes afford an optical means for investigation of mPTP gating in living cells. In addition, the presence of spontaneous superoxide flashes provides evidence for physiological mPTP activity in quiescent cells.

### Example 7

### Electron Transport Chain Activity is Required for Superoxide Flash Production

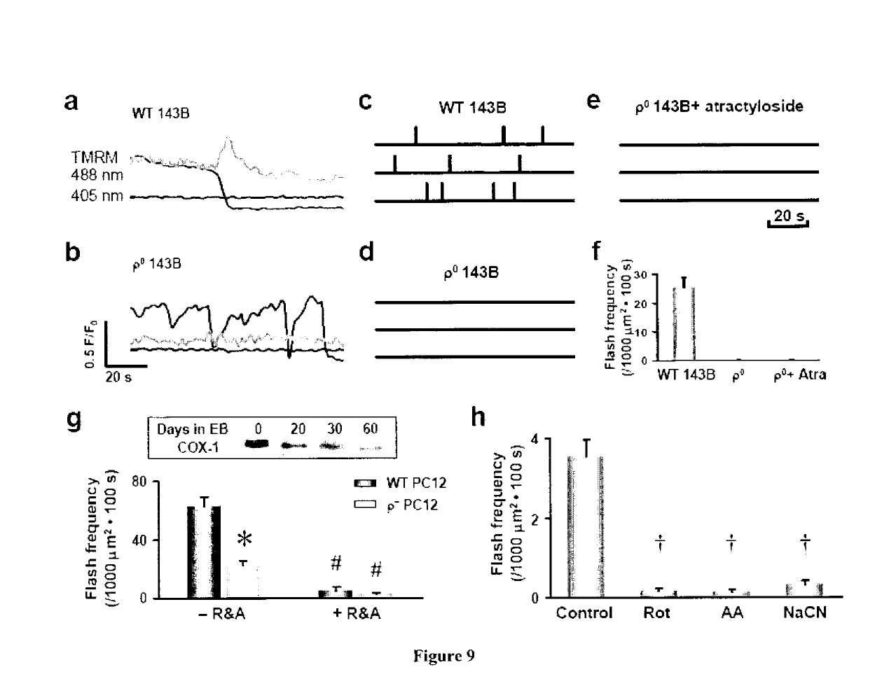

The role of the ETC in superoxide flash production was established by complete depletion of mitochondrial DNA, as in ρ° 143B TK-human osteosarcoma cells. In this cell model, mitochondrial respiration is abrogated altogether due to lack of crucial ETC proteins coded by mitochondrial DNA (King et al., Science, 246: 500-503, 1989). It was found that superoxide flashes are absent in ρ° cells (n=20, FIGS. 9b,d), and cannot be rescued even by the mPTP activator atractyloside (FIGS. 9e-f). In wild type 143B cells, however, robust superoxide flash activity was observed at a rate of 25±4 (events per 1000 μm2 cell area per 100 s, n=21. FIGS. 9a,c). It follows that superoxide flash production requires ETC activity, which presumably supplies the O₂— that fuels the flash via the electron leakage mechanism.

Constitutive electron leakage from the ETC sets basal levels of ROS signals (e.g., O₂.⁻, H₂O₂ and OH) that can directly or indirectly modulate mPTP activity (Vercesi et al., Biosci. Rep., 17: 43-52, 1997; Turrens, J. Physiol., 552: 335-344, 2003). Under this scenario, it was investigated whether ETC activity is an intrinsic regulator of the flash production by creating an ETC defective (ρ−) cell model following ethidium bromide (200 ng/ml for 60 days) inhibition of mitochondrial DNA replication in rat PC12 pheochromocytoma cells. Partial deprivation of mitochondrial DNA in ρ− PC12 cells resulted in a parallel decrease in both cytochrome C oxidase subunit I (COX-1) expression (70% of control) and the incidence of superoxide flashes (60% of control, from 63±6 to 23±3 events per 1000 μm2 cell area per 100 s, n=37-46, FIG. 9g). The linkage between ETC activity and superoxide flash periodicity was reinforced by use of classic mitochondrial ETC inhibitors. Rotenone (5 μM), antimycin A (AA, 5 μg/ml) and sodium cyanide (NaCN, 5 mM), which block ETC at complexes I, III and IV, respectively, all virtually abolished the occurrence of superoxide flashes in cardiac myocytes as well as ρ− PC 12 cells (FIGS. 9g-h). It is noteworthy that AA-induced decreased superoxide flashes in sharp contrast to the previous finding that AA increases cellular ROS production when measured with the H₂O₂ indicator dichlorodihydrofluorescein (DCF, Aon et al., J. Biol. Chem. 278: 44735-44744, 2003). This apparent discrepancy may be reconciled by the fact that DCF does not discriminate between intra- and extra-mitochondrial matrix ROS signals, and AA inhibits electron transfer from the outer (Qo) to inner (Qi) center of complex III, decreasing matrix O₂.⁻ production while facilitating production of O₂.⁻ (membrane-impermeable) and H₂O₂ (membranepermeable but cpYFP-insensitive) toward the cytosol (Turrens, J. Physiol., 552: 335-344, 2003).

### Example 8

### Frequency-Dependent Modulation of Superoxide Flashes in Cardiac Myocytes During Hypoxia and Reoxygenation

Oxidative stress and aggravated ROS production contribute to the pathogenesis of a number of clinically distinct disorders including neurodegeneration (e.g. Alzheimer's disease), tissue inflammation, hypertension, atherosclerosis, diabetes, and cancer (Andersen, Nat. Med., 10: S18-S25, 2004; Dhalla et al., J. Hypertens., 18: 655-673, 2000; Klaunig and Kamendulis, Annu. Rev. Pharmacol, Toxicol., 44:239-267, 2004). Since flashes are triggered by mPTP activity that is itself sensitive to ROS (Vercesi et al., Biosci. Rep., 17: 43-52, 1997; Turrens, J. Physiol., 552: 335-344, 2003), the frequency of superoxide flashes may vary during stress or disease, and may therefore serve as a biomarker of oxidative stress such as those in ischemia-reperfusion. Sustained hypoxic treatment (95% N₂ and 5% CO₂ for 6 hrs) depressed

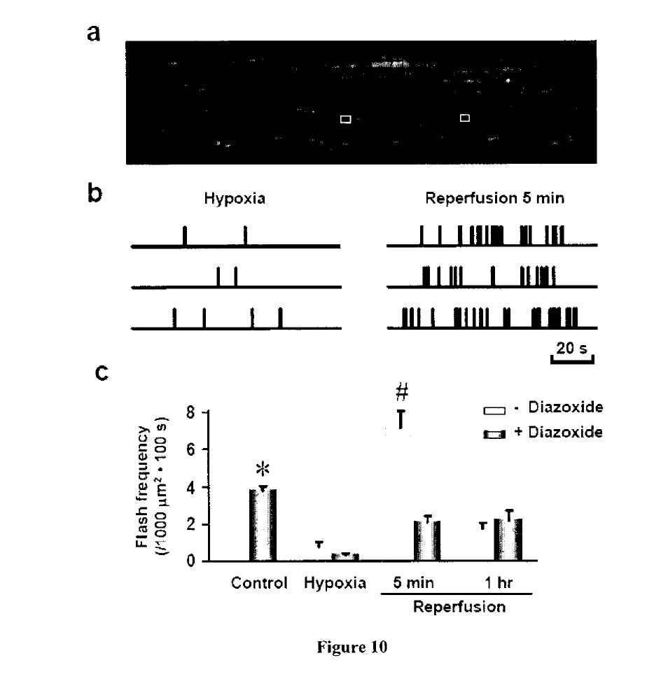

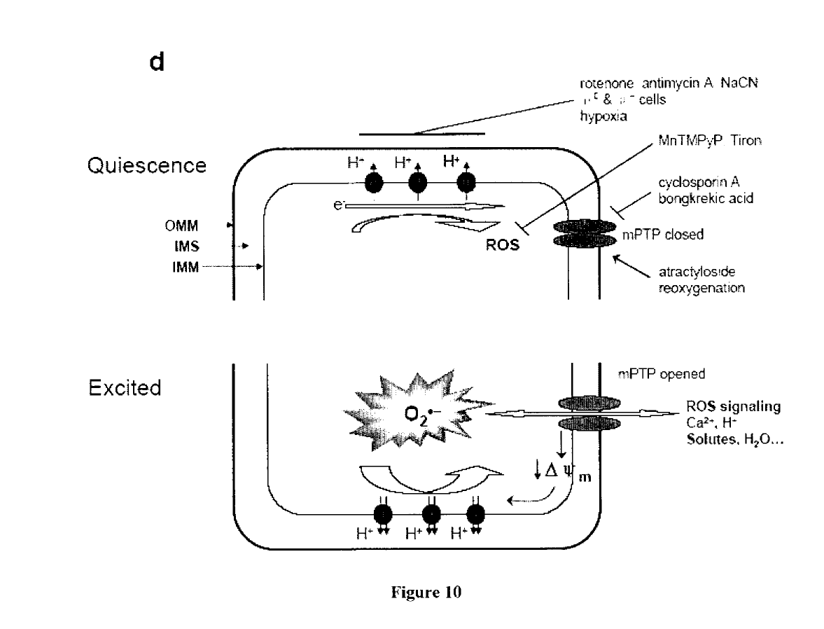

superoxide flash production by 70% (FIG. 10c), further indicating that mitochondrial respiration is a critical determinant of superoxide flash frequency. Shortly after reoxygenation (˜5 min), in the period vulnerable to oxidative damage (Weiss et al., Circ. Res., 93: 292-301, 2003; Garlick et al., Circ. Res., 61: 757-760, 1987), a rebound flurry of superoxide flash activity was observed that was 1.9-fold higher compared to normoxia controls. Two-dimensional mapping and temporal diaries of flash activity show a random distribution of superoxide flashes over space and time (FIGS. 10a-b). Spatiotemporal summation of these superoxide flashes may contribute significantly to enhanced ROS signalling under these conditions. Over time, superoxide flash activity eventually receded to a level below that of normoxia controls (FIG. 10c), consistent with an irreversible mitochondrial damage inflicted by the hypoxia and reperfusion procedure. Previous studies have shown that ischemia-reperfusion associated damages is alleviated by preconditioning cells with diazoxide, an opener of mitochondrial ATP-sensitive potassium channels. Likewise, diazoxide pretreatment (30 μM added 20 min prior to hypoxia) effectively protected the cells from the rebound flurry of superoxide flash activity (FIG. 10c).

Overall, mt-cpYFP enables real-time measurement of robust single mitochondrion superoxide bursts that arise from mTPT openings and ETC activity under physiological conditions across a wide range of cell types. In quiescence, constitutive electron leakage from the ETC plays a central role in setting the physiological level of ROS (e.g. O₂.⁻, H₂O₂, .OH) production that triggers infrequent, stochastic openings of the mPTP. Upon mPTP opening, the ETC-linked O₂.⁻ producing machinery is excited concurrently with the abolition of electrical and chemical gradients across the inner membrane, the further activation of the ETC, and perhaps mitochondrial swelling due to water movement. This gives rise to a burst of matrix O₂.⁻ production that is visualized as a superoxide flash in a single mitochondrion. (FIG. 10d).

What is claimed is:

1. A method for monitoring superoxide formation in a cell, comprising
providing a protein probe comprising an amino acid sequence with at least 80% sequence identity to SEQ ID NO. 1 to the cell; and
measuring the fluorescence of the protein probe, wherein a change in fluorescence of the probe correlates with a change in superoxide formation.

2. The method for monitoring superoxide formation in a cell of claim 1, wherein the protein probe is operatively attached to a targeting sequence that causes the protein probe to localize to a specific cellular compartment.

3. The method for monitoring superoxide formation in a cell of claim 2, wherein the specific cellular compartment is selected from the group consisting of:
mitochondria, the cytoplasm, the Golgi, the endoplasmic/sarcoplasmic reticulum, the nucleus, peroxisomes, and the plasma membrane.

4. The method for monitoring superoxide formation in a cell of claim 1, wherein the protein probe comprises one or more amino acids residues selected from the group consisting of:
D13, H13, A28, V28, G40, S40, F68, Y68, H68, T68, L158, C160, G177, S177, T177, A177, Y178, W178, H178, G179, L180, V180, K181, Q181 and C182.

5. The method for monitoring superoxide formation in a cell of claim 1, further comprising;
contacting the cell with the therapeutic agent while continuing to measure the fluorescence of the protein probe,
wherein a change in fluorescence of the probe correlates with a change in superoxide formation inside the cell, and further correlates to an effect of the therapeutic agent on superoxide formation inside the cell.

6. A method for monitoring superoxide formation in a cell, comprising
providing a protein probe comprising an amino acid sequence with at least 80% sequence identity to SEQ ID NO. 3 to the cell; and
measuring the fluorescence of the protein probe, wherein a change in fluorescence of the probe correlates with a change in intracellular superoxide formation.

7. The method for monitoring superoxide formation in a cell of claim 6, wherein the protein probe is operatively attached to a targeting sequence that causes the protein probe to localize to a specific cellular compartment.

8. The method for monitoring superoxide formation in a cell of claim 7, wherein the specific cellular compartment is selected from the group consisting of:
mitochondria, the cytoplasm, the Golgi, the endoplasmic/sarcoplasmic reticulum, the nucleus, peroxisomes, and the plasma membrane.

9. The method for monitoring superoxide formation in a cell of claim 7, wherein the protein probe comprises one or more amino acids residues selected from the group consisting of:
D177, H177, A192, V192, S204, S204, F232, Y232, H232, T232, L75, C77, G94, S94, T94, A94, Y95, W95, H95, G96, L97, V97, K98, Q98 and C99.

10. A fluorescent protein probe for monitoring superoxide formation inside a cell, wherein the protein probe comprises an amino acid sequence with at least 80% sequence identity to SEQ ID NO. 1.

11. The fluorescent protein probe for monitoring superoxide formation inside a cell of claim 10, wherein the protein probe comprises one or more amino acids residues selected from the group consisting of:
D13, H13, A28, V28, G40, S40, F68, Y68, H68, T68, L158, C160, G177, S177, T177, A177, Y178, W178, H178, G179, L180, V180, K181, Q181 and C182.

12. A fluorescent protein probe for monitoring superoxide formation inside a cell, wherein the protein probe comprises an amino acid sequence with at least 80% sequence identity to SEQ ID NO. 3.

13. The fluorescent protein probe for monitoring superoxide formation inside a cell of claim 12, wherein the protein probe comprises one or more amino acids residues selected from the group consisting of:
D177, H177, A192, V192, G204, S204, F232, Y232, H232, T232, L75, C77, G94, S94, T94, A94, Y95, W95, H95, G96, L97, V97, K98, Q98 and C99.

14. A nucleic acid comprising a nucleic acid sequence that encodes an amino acid sequence with at least 80% sequence identity to SEQ ID NO. 1.

15. A nucleic acid comprising a nucleic acid sequence that encodes an amino acid sequence with at least 80% sequence identity to SEQ ID NO. 3.

16. A nucleic acid comprising the nucleic acid sequence with at least 80% sequence identity to SEQ ID NO. 4.

17. A cell capable of expressing a protein probe comprising an amino acid sequence with at least 80% sequence identity to SEQ ID NO. 1 or SEQ ID NO. 3.

18. A non-human organism comprising one or more cells capable of expressing a protein probe comprising an amino acid sequence with at least 80% sequence identity to SEQ ID NO. 1 or SEQ ID NO. 3.

19. A method for predicting progression of a disease based on a change in intracellular superoxide formation, comprising:
providing one or more cells capable of expressing a protein probe comprising an amino acid sequence with at least 80% sequence identity to SEQ ID NO. 1 or SEQ ID NO. 3;
causing the plurality of cells to develop one or more characteristics of the disease; and
measuring the change in the intracellular superoxide formation of one or more of the cells,
wherein the change intracellular superoxide formation is indicative of the progression of the disease state.

20. A method for predicting progression of a disease based on a change in intracellular superoxide formation, comprising:
providing an organism having one or more cells capable of expressing a protein probe comprising an amino acid sequence with at least 80% sequence identity to SEQ ID NO. 1 or SEQ ID NO. 3;
causing the organism to develop one or more characteristics of the disease; and
measuring the change in intracellular superoxide formation of one or more cells of the organism,
wherein the change in intracellular superoxide formation is indicative of the progression of the disease state.


SEQUENCE LISTING (4 sequences)

SEQ ID NO: 1 - protein, 258 aa, Artificial
  Sequence modified from U.S. published patent application 20050208624
MRSGIGSAGYNSDNVYIMADKQKNGIKANFKIRHNIEDGGVQLADHYQQNTPIGDGPVLLPDNHYLSFQSALSKDPNEKRDHMVLLEFVTAAGITLGMDELYKVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNGT

SEQ ID NO: 2 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 3 - protein, 269 aa, Artificial
  Non-permuted version of cpYFP containing linker sequences
MLSLRQSIRFFKRSGIGSAGVDGGSGGTGVSKGEELFTGVVPILVELDGDVNGHKFSVSGEGEGDATYGKLTLKLICTTGKLPVPWPTLVTTFGYGLKCFARYPDHMKQHDFFKSAMPEGYVQERTIFFKDDGNYKTRAEVKFEGDTLVNRIELKGIDFKEDGNILGHKLEYNYNSDNVYIMADKQKNGIKANFKIRHNIEDGGVQLADHYQQNTPIGDGPVLLPDNHYLSFQSALSKDPNEKRDHMVLLEFVTAAGITLGMDELYKGT

SEQ ID NO: 4 - DNA, 777 bp, Artificial
  Nucleic acid modified from U.S. published patent application 2005-0208624
atgcgcagcggcatcgggtctgcaggctacaacagcgacaacgtctatatcatggccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcttccagtccgccctgagcaaagaccccaacgagaagcgcgatcacatggtcctgctggagttcgtgaccgccgccgggatcactctcggcatggacgagctgtacaaggtcgacggtggcagcggtggcaccggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctcatctgcaccaccggcaagctgcccgtgccctggcccaccctcgtgaccaccttcggctacggcctgaagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtacctaa